(19) United States
(12) Patent Application Publication
(10) Pub. No.: US 2025/0241783 A1
(43) Pub. Date: Jul. 31, 2025

(54) ANTI-SNORE MOUTHPIECE

(71) Applicant: Wisdom Depot LLC, Roslyn, NY (US)

(72) Inventor: Cheng-yi Shih, Roslyn, NY (US)

(21) Appl. No.: 19/042,692

(22) Filed: Jan. 31, 2025

Related U.S. Application Data
(60) Provisional application No. 63/627,574, filed on Jan. 31, 2024.

(51) Int. Cl. A61F 5/56

(52) U.S. Cl. CPC A61F 5/566

(57) ABSTRACT

According to an aspect of an embodiment, a mouthpiece may include a first piece, a second piece, and a film. The first piece may be configured to engage upper teeth of a user. In some embodiments, the first piece may have a first end and a second end. The second piece may be configured to engage lower teeth of the user, and the second piece may be attached to the second end of the first piece. In some embodiments, the film may be coupled between the first piece and the second piece. The film may be configured to control airflow through the mouthpiece.

### CROSS-REFERENCE TO RELATED APPLCIATIONS

This patent application claims priority to U.S. Provisional Application 63/627,574 filed on Jan. 31, 2024, which is incorporated herein by specific reference in its entirety.

### FIELD

The embodiments discussed in the present disclosure are related to a mouthpiece configured to help reduce snoring.

### BACKGROUND

In general, snoring occurs as the airflow through the mouth and nose is at least partially obstructed during sleep, causing the tissues in the throat to vibrate. Such obstruction and the snoring may be due to various factors such as nasal congestion, relaxation of throat muscles, obesity, sleep position, etc. Different methods or systems may be used to help reduce snoring. For example, a mouthpiece may help alleviate snoring by repositioning the lower jaw and the tongue of a user to keep the airway open.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of an embodiment, a mouthpiece may include a first piece, a second piece, and a film. The first piece may be configured to engage upper teeth of a user. In some embodiments, the first piece may have a first end and a second end. The second piece may be configured to engage lower teeth of the user, and the second piece may be attached to the second end of the first piece. In some embodiments, the film may be coupled between the first piece and the second piece. The film may be configured to control airflow through the mouthpiece.

The object and advantages of the embodiments will be realized and achieved at least by the elements, features, and combinations particularly pointed out in the claims. It is to be understood that both the foregoing general description and the following detailed description are exemplary and explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1A includes a front view of an example mouthpiece having a film;

FIG. 1B includes a closed view of the mouthpiece of FIG. 1A;

FIG. 1C includes a rear view of the mouthpiece of FIG. 1A;

FIG. 1D includes a side view of the mouthpiece of FIG. 1A;

FIG. 1E includes a front view of the mouthpiece of FIG. 1A having extruded surfaces;

FIG. 1F includes a closed view of the mouthpiece of FIG. 1A having extruded surfaces;

FIG. 1G includes a front of the mouthpiece of FIG. 1A having one or more holes;

FIG. 1H includes a front view of the mouthpiece of FIG. 1A having one or more ridges;

FIG. 1I includes a front of the mouthpiece of FIG. 1A having a handle;

FIG. 2A includes a side view of another example mouthpiece having a film;

FIG. 2B includes a side view of the mouthpiece of FIG. 2A having a handle;

FIG. 2C includes a rear view of the mouthpiece of FIG. 2A; and

FIG. 3 illustrates a flow chart of an example method of reducing snoring.

The elements and components in the figures can be arranged in accordance with at least one of the embodiments described herein, and which arrangement may be modified in accordance with the disclosure provided herein by one of ordinary skill in the art.

### DESCRIPTION OF EMBODIMENTS

Snoring may be caused by various reasons such as mouth breathing, nasal congestion, sleep position, obesity, sleep apnea, relaxed throat muscles, etc. Snoring may cause several negative effects on a person such as poor sleep quality, daytime fatigue, increased risk of heart disease, diabetes, stroke, relationship strain, reduced oxygen supply, etc.

Different devices or system may be used to reduce snoring. For example, mandibular advancement devices (MADs), continuous positive airway pressure (CPAP) machine, positional therapy devices, anti-snoring pillows, tongue stabilizing devices (TSDs), etc. Different devices may work in different ways to help reduce snoring. In particular, a mouthpiece (a type of mandibular advancement device (MAD)) may help reduce snoring by advancing the lower jaw, preventing tongue collapse, reducing airway vibrations, etc.

Some existing mouthpieces may rely on pulling the lower jaw forward to prevent blocking the airway during sleep. However, such a mechanism may cause the user to wake up with a sore lower jaw and may need to adjust the jaw back to its original position after taking off the mouthpiece.

Another existing mouthpiece may rely on tongue suction mechanism to pull the tongue of the user forward (e.g., toward the lips) to prevent the tongue and the lower jaw from falling backward and blocking the airway. However, such suction mechanism may not work throughout the night and the mouthpiece may make swallowing saliva difficult. Additionally, such existing mouthpieces may not be effective as the user opens mouth during sleep.

According to one or more embodiments of the present disclosure, a mouthpiece may be configured with a film coupled or located between a first piece and a second piece. For example, the first piece may be configured to mate with or cover upper teeth of a user, and the second piece may be configured to mate with or cover lower teeth of the user. The film may be coupled between the first piece and the second piece, and the film may be configured to control the airflow through the mouthpiece.

The film may be configured to move or fold as the user opens and closes mouth, such that the film continues to control the airflow whether the mouthpiece or the mouth of the user is open or closed. In the present disclosure, a reference to the mouthpiece being closed may refer to the first piece and the second piece coming together, and a reference to the mouthpiece being open may refer to the first piece and the second piece being apart. The film may be configured to help prevent the lower jaw from falling downward during sleep and hold the jaw in a naturally aligned position with the upper jaw without forcing the lower jaw forward into the underbite position. Such configuration may provide improved comfort for the user while controlling airflow.

The mouthpiece according to one or more embodiments of the present disclosure may provide improvements with respect to existing approaches. For example, the film may help block airflow through the mouth to help user breathe through the nose, which may help reduce snoring. The film may also help the mouthpiece to stay in the mouth during sleep. For example, the tension in the film may cause the film to expand as the user opens the mouth, such that the film continuously covers the mouth, and the mouthpiece stays in position. Additionally, the tension of the film may be used to exercise jaw muscles via the closing and opening of the mouth (e.g., biting down and up).

Embodiments of the present disclosure will be explained with reference to the accompanying drawings.

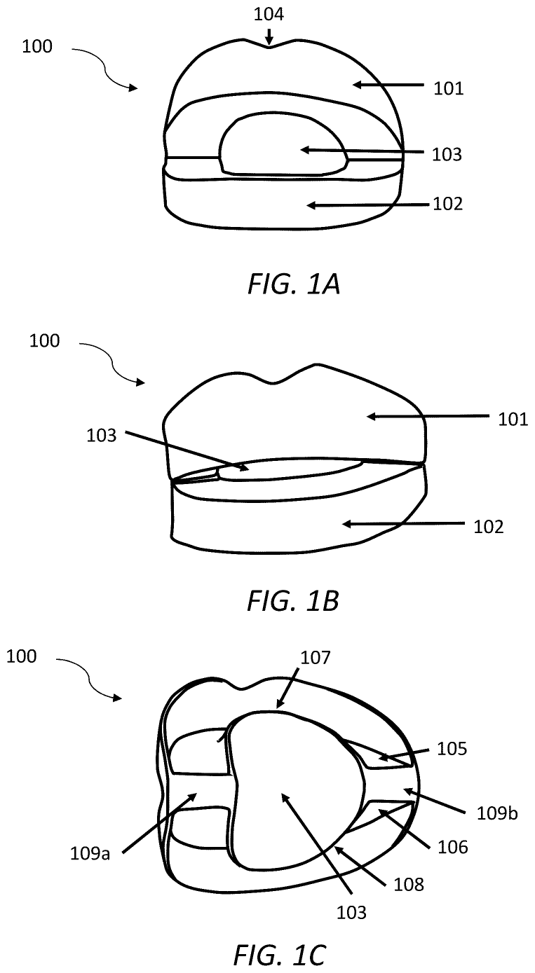

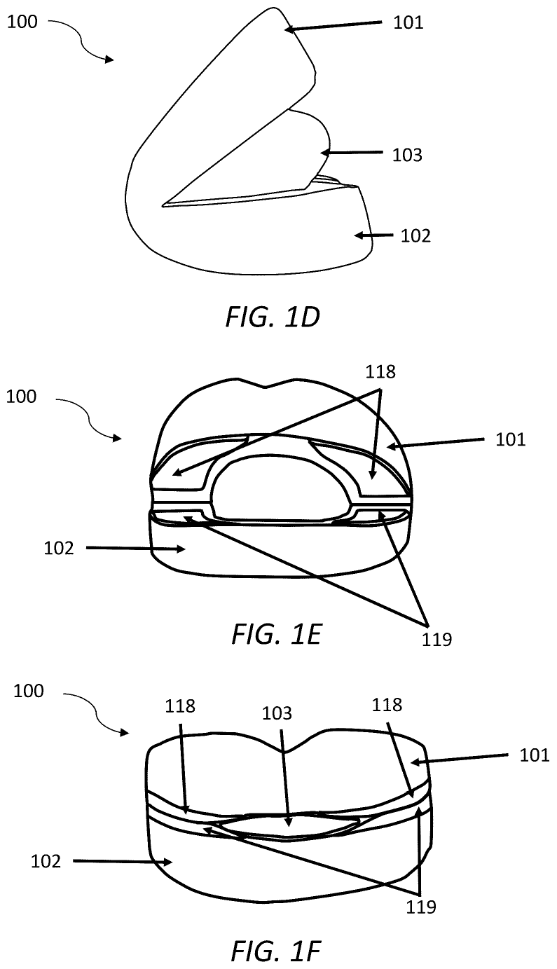

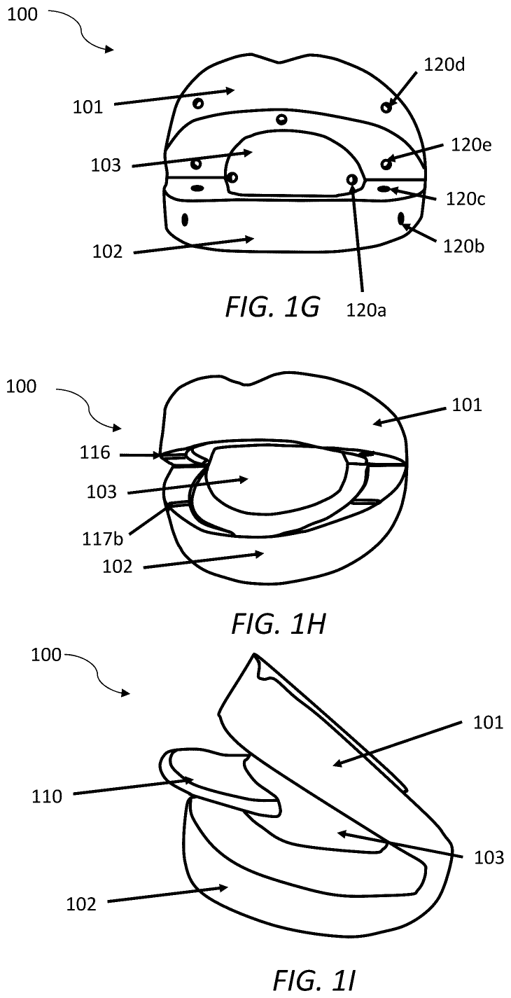

FIGS. 1A-1I illustrates an example mouthpiece 100, in accordance with one or more embodiments of the present disclosure, in varying perspectives. For example, FIG. 1A illustrates a front view of the mouthpiece 100. In some embodiments, the mouthpiece 100 may include a first piece 101, a second piece 102, and a film 103. In some embodiments, the first piece 101 may include an indentation 104, configured to indicate the first piece to help users identify between the first piece 101 and the second piece 102. While the indentation 104 is illustrated as being in the central area of the first piece 101, the indentation 104 may be located anywhere within the first piece. Additionally or alternatively, the first piece 101 or the second piece 102 may include any other suitable types of identifiers, such as markings, colors, etc.

In some embodiments, the mouthpiece 100 may be configured to be inserted into a mouth of a user. The mouthpiece 100 may be configured to cover or mate with teeth of the user, such that the mouthpiece may stay in place inside the user's mouth. For example, in some embodiments, the first piece 101 may be configured to mate with or cover the upper teeth of the user, and the second piece 102 may be configured to mate with or cover the lower teeth of the user. The first piece 101 and the second piece 102 may mate with the upper teeth and the lower teeth, respectively, such that the mouthpiece is generally stays in place with respect to the upper teeth and the lower teeth. For example, the first piece 101 may include a first teeth tray 105 configured to receive the upper teeth of the user therein. The second piece 102 may include a second teeth tray 106 configured to receive lower teeth of the user therein. In some embodiments, the first piece 101 may be referred to as an upper piece, and the second piece 102 may be referred to as a lower piece. In some embodiments, the first piece 101 and/or the second piece 102 may be configured to cover at least a part of the lips. For example, the first piece 101 may be configured to cover at least a part of the upper lip of the user wearing the mouthpiece 100. The second piece 102 may be configured to cover at least a part of the lower lip of the user wearing the mouthpiece 100. For example, in some embodiments, the first piece 101 may cover the upper teeth and at least a part of the upper lips, and the second piece 102 may cover the lower teeth and at least a part of the lower teeth.

In some embodiments, the first piece 101 and the second piece 102 may respectively have a first end and a second end. The first end may be an end or a side facing the lips of the user when the mouthpiece 100 is inserted into the user's mouth, and the second end may be an end or a side facing the throat of the user when the mouthpiece 100 is inserted into the user's mouth. In some embodiments, the first piece 101 and the second piece 102 may be connected via respective second ends. In some embodiments, the first piece 101 and the second piece may be connected via a first hinges 109a and a second hinge 109b as illustrated in FIG. 1C. In these and other embodiments, the first piece 101 and the second piece 102 may be connected in a manner such that there is an opening between the first piece 101, the second piece 102, the first hinge 109a and the second hinge 109b. In these and other embodiments, the opening between the first hinge 109a and the second hinge 109b may allow the tongue of the user to rest in place without being blocked off by the connection between the first piece 101 and the second piece 102.

In some embodiments, the first piece 101 and the second piece 102 may be manufactured as a single piece of item. For instance, the first piece 101, the second piece 102, the first hinge 109a, and the second hinge 109b may be manufactured as a single piece.

In some embodiments, the film 103 may be coupled between the first piece 101 and the second piece 102, such that the film may cover the opening. In some embodiments, the film may include a curvature such that the film 103 does not interfere with the tongue of the user. For example, FIG. 1D illustrates a side view of the mouthpiece 100, showing the curvature of the film 103. In some embodiments, the curvature of the film 103 may permit the film 103 to fold between the first piece 101 and the second 102 as the user opens and closes the mouth while wearing the mouthpiece 100. For example, FIG. 1A illustrates the mouthpiece 100 open. In the present disclosure, a reference to the mouthpiece 100 or the mouth of the user being open may include a reference to the first piece 101 and the second piece 102 being apart at a certain distance.

FIG. 1B illustrates the mouthpiece 100 closed, in which the first piece 101 and the second piece 102 are moved closer together. As the mouthpiece 100 moves from a first position illustrated in FIG. 1A to a second position illustrated in FIG. 1B, the film may fold, as illustrated in FIG. 1B, such that the film does not break or interfere with the movement of the mouthpiece 100. In some embodiments, the film 103 may act as a spring mechanism between the first piece 101 and the second piece 102. For example, the film 103 may have a tendency to return to or expand to a first position (e.g., as illustrated in FIG. 1A), such that the film 103 helps the mouthpiece 100 return to the first position. In these and other embodiments, the film 103 may help the mouthpiece 100 adjust to the size of the user's mouth, such that the mouthpiece 100 may stay in the user's mouth during sleep or movement.

In some embodiments, the curvature and/or the thickness of the film 103 may be adjusted to required different levels of pressure to move between the first position and the second position. In these and other embodiments, the different levels of pressure may help tighten soft tissues in a mouth of the user to open airway.

In some embodiments, the mouthpiece 100 may include one or more extruded surfaces. For example, in some embodiments, the first piece 101 may include first extruded surfaces 118, and the second piece 102 may include second extruded surfaces 119, as illustrated in FIG. 1E. The first extruded surfaces 118 and the second extruded surfaces 119 may be configured to provide support or padding for the first piece 101 and the second piece 102. For example, as the user bites down or closes the mouthpiece 100, the first extruded surfaces 118 and the second extruded surfaces 119 may contact to help prevent the first piece 101 and the second piece 102 form contacting directly. Such contact between the first extruded surfaces 118 and the second extruded surfaces 119 may provide additional support for the first piece 101 and the second piece 102.

In some embodiments, the first extruded surfaces 118 and the second extruded surfaces 119 may be configured such that when the mouthpiece is closed, the extruded surfaces and the film 103 in the folded position are leveled. For example, as illustrated in FIG. 1F, as the first extruded surfaces 118 and the second extruded surfaces 119 mate or contact, the film 103, as folded, may be level with the extruded surfaces. In these and other embodiments, the extruded surfaces (e.g., the first extruded surfaces 118 and the second extruded surfaces 119) may be configured such that the extruded surfaces do not overlap with the film 103.

In some embodiments, the film 103 may be configured to block and/or control the airflow through the opening or the mouth of the user. For example, the film 103 may seal the opening such that no air goes through the opening (e.g., between the first piece 101 and the second piece 102) whether the mouthpiece 100 is open or closed. In these and other embodiments, the film 103 may block or reduce the airflow to help reduce or prevent uvula and the back of the tongue from vibrating, which are common causes of snoring. Additionally or alternatively, the film 103 may may cause the user to breathe through nose, instead of the mouth.

In some embodiments, the mouthpiece may be used as a complement continuous positive airway pressure (CPAP) and/or bilevel positive airway pressure (BiPAP) usage with a nasal mask or nasal pillow mask. For example, the mouthpiece 100 may help prevent discomfort such as abdominal discomfort and bloating due to aerophagia (e.g., air swallowing) during sleep by reducing air flowing through the mouth and into the digestive system. Further, the mouthpiece 100 may help prevent air leakage through the mouth to improve the effectiveness of CPAP and BiPAP.

In some embodiments, the mouthpiece 100 may include one or more holes 120-120e configured to control the airflow through the mouthpiece 100. For example, the holes 120 may be configured to act as air vents to allow controlled amount of air through the mouthpiece 100. For example, the air flow through the mouthpiece 100 may be controlled based on shape, size, and/or location of the holes 120.

In some embodiments, the holes 120 may be placed anywhere within the mouthpiece 100. For example, a first hole 120a is located on the film 103 while the holes 120b and 120c are placed on the second piece, and the holes 120d and 120e are placed on the first piece 101. The number, shape, location, and size of the holes 120 are shown for illustrative purposes only. Any suitable number of holes 120 may be located at any suitable locations within the mouthpiece 100 having any suitable shapes and sizes. In some embodiments, the holes 120 may have varying sizes or shapes. For example, a hole placed on the film 103 may be smaller than a hole placed on the first piece. The holes 120 may help the users who have hard time breathing through the nose.

In some embodiments, the holes 120 may also help movement or transfer of saliva to the lips past the mouthpiece 100. The movement of saliva may help moisturize the lips.

In some embodiments, the mouthpiece 100 may include one or more ridges configured to help align and/or interlock the first piece 101 and the second piece 102 in the closed position (e.g., as illustrated in FIG. 1B). For example, FIG. 1G illustrates a first ridge 116 and a second ridge 117. In some embodiments, the first ridge 116 may be located on the first piece 101 on the surface facing the second piece 102, and the second ridge 117 may be located on the second piece 102 facing the first piece 101. In these and other embodiments, the first ridge 116 and the second ridge 117 may be configured to mate or interlock as the mouthpiece 100 closes to provide interlocking and additional support to hold the first piece 101 and/or the second piece 102 in position. In some embodiments, the mouthpiece 100 may include any suitable numbers of the ridges located at any suitable locations. Additionally, the ridges may include any suitable shapes. For example, the ridges may be extruded outward or inward.

In some embodiments, the first piece 101, the second piece 102, and the film 103 may be manufactured as a single item or piece. For example, the first piece 101, the second piece 102, and the film 103 may be manufactured such that the first piece 101, the second piece 102, and the film are inseparable. In these and other embodiments, the mouthpiece 100 may be manufactured without molding, which may help improve strength and easier maintenance of the mouthpiece 100. In other embodiments, the first piece 101, the second piece 102, and/or the film 103 may be manufactured separately and built and/or connected together to form the mouthpiece 100.

In some embodiments, different parts of the mouthpiece 100 may be manufactured from the same type of material. For example, in some embodiments, the first piece 101, the second piece 102, and the film 103 may be manufactured using the same type of material. In some embodiments, the material of the mouthpiece may include silicone or a silicone-like material. The silicone may help improve safety of the mouthpiece 100 as the silicone may help reduce sharp or rough edges that may cause damages to the user's mouth. Additionally, the silicone may reduce chances of parts of the mouthpiece breaking, which may lead to chocking or other dangers. In some embodiments, the mouthpiece 100 may be manufactured using any other suitable types of materials. For example, the mouthpiece 100 may be manufactured using acrylic, thermoplastic materials, silicone, polycarbonate, acetal, thermoplastic elastomer, soft foam inserts, etc. In these and other embodiments, the single-piece design may improve cleaning of the mouthpiece 100

In some embodiments, different parts of the mouthpiece 100 may be manufactured using different materials. For example, the first teeth tray 105 and second teeth tray 106 may be manufactured using moldable materials that may be molded to provide custom fit for the different users, while the first piece 101, the second piece 102, and the film 103 are manufactured using heat-resistance materials to help maintain shape or form.

In some embodiments, the mouthpiece 100 may include a handle 110 configured to help improve extraction of the mouthpiece form the user's mouth, as illustrated in FIG. 1I. For example, the user may hold and pull the handle 110 to remove the mouthpiece 100 from the user's mouth. In some embodiments, the handle 110 may be placed on the film 103. In these and other embodiments, the handle 110 may be located such that the handle 110 does not interfere with movement of the film 103. For example, the handle 110 may not interfere with the folding and/or expanding of the film 103 as the mouthpiece 100 opens and closes. In these and other embodiments, the thickness of the handle 110 may be determined such that the handle 110 does not prevent the mouthpiece 100 from closing.

In some embodiments, the handle 110 may be located at other locations of the mouthpiece 100. For example, the handle 110 may be located on the first piece 101 or the second piece 102. In these and other embodiments, the handle 110 may be shaped and sized such that the handle 110 does not affect operations of the mouthpiece 100 or provide discomfort for the user.

In some embodiments, the handle 110 may be a part of the mouthpiece 100. For example, the handle 110 may be manufactured as a single piece including the first piece 101, the second piece 102, the film 103, and the handle 110. For example, the mouthpiece 100 may be manufactured using an injection-molding method in which the mold includes the first piece 101, the second piece 102, the film 103, and the handle 110.

In some embodiments, the handle 110 may be removably attached to the mouthpiece 100. For example, the handle 110 may be attached to the film 103 using adhesives such as resin, silicone, epoxy, etc. In these and other embodiments, the adhesives may be food-grade or non-toxic such that the adhesives are safe to use on in the mouthpiece 100. The handle 110 may be removed from the mouthpiece 100 by removing the adhesives. In other embodiments, any other suitable means of removably attaching the handle 110 to the mouthpiece 100 may be used.

Modifications, additions, or omissions may be made to the mouthpiece 100 without departing from the scope of the present disclosure. For example, in some embodiments, the mouthpiece 100 may include any number of other components that may not be explicitly illustrated or described.

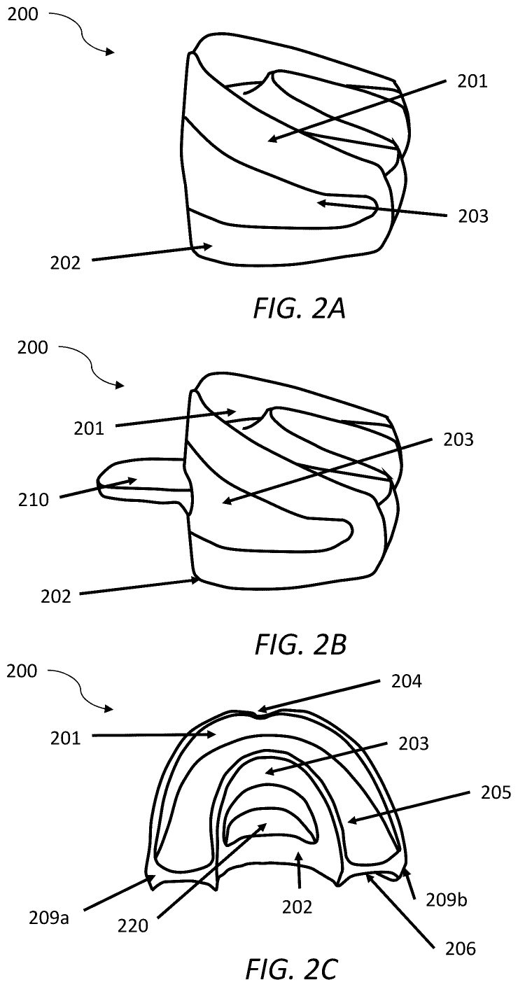

FIGS. 2A-2C illustrates another example mouthpiece 200, in accordance with one or more embodiments of the present disclosure, in varying perspectives. In some embodiments, the mouthpiece 200 may include a first piece 201, a second piece 202, and a film 203. In some embodiments, the first piece 201 may include an indentation 204, configured to indicate the first piece 201 to help users identify between the first piece 201 and the second piece 202. While the indentation 204 is illustrated as being in the central area of the first piece 201, the indentation 204 may be located anywhere within the first piece. Additionally or alternatively, the first piece 201 or the second piece 202 may include any other suitable types of identifiers, such as markings, colors, etc.

In some embodiments, the first piece 201 and the second piece 202 may correspond to the first piece 101 and the second piece 102 of FIGS. 1A-1I. For example, the descriptions of the first piece 101 and the second piece 102 may be applicable to the first piece 201 and the second piece 202.

For example, in some embodiments, the mouthpiece 200 may be configured to be inserted into a mouth of a user. The mouthpiece 200 may be configured to cover or mate with teeth of the user, such that the mouthpiece may stay in place inside the user's mouth. For example, in some embodiments, the first piece 201 may be configured to mate with or cover the upper teeth of the user, and the second piece 202 may be configured to mate with or cover the lower teeth of the user. The first piece 201 and the second piece 202 may mate with the upper teeth and the lower teeth, respectively, such that the mouthpiece is generally stays in place with respect to the upper teeth and the lower teeth. For example, the first piece 201 may include a first teeth tray 205 configured to receive the upper teeth of the user therein. The second piece 202 may include a second teeth tray 206 configured to receive lower teeth of the user therein. In some embodiments, the first piece 201 may be referred to as an upper piece, and the second piece 102 may be referred to as a lower piece.

In some embodiments, the first piece 201 and the second piece 202 may respectively have a first end and a second end. The first end may be an end or a side facing the lips of the user when the mouthpiece 200 is inserted into the user's mouth, and the second end may be an end or a side facing the throat of the user when the mouthpiece 200 is inserted into the user's mouth. In some embodiments, the first piece 201 and the second piece 202 may be connected via respective second ends. In some embodiments, the first piece 201 and the second piece may be connected via a first hinges 209a and a second hinge 209b as illustrated in FIG. 2C. In these and other embodiments, the first piece 201 and the second piece 202 may be connected in a manner such that there is an opening between the first piece 201, the second piece 202, the first hinge 209a and the second hinge 209b. In these and other embodiments, the opening between the first hinge 209a and the second hinge 209b may allow the tongue of the user to rest in place without being blocked off by the connection between the first piece 201 and the second piece 202.

In some embodiments, the film 203 may be coupled between the first piece 201 and the second piece 202, such that the film 203 is connected to exterior walls (e.g., walls on the first end) of the first piece 201 and the second piece 202. In some embodiments, the mouthpiece 200 may include a cavity 220 at the back of the mouthpiece 200 or at the second ends of the first piece 201 and the second piece 202, as illustrated in FIG. 2C. The cavity 220 may provide space for the film 203 to fold inward toward the second end as the first piece 201 and the second piece 202 move toward each other (e.g., closing the mouthpiece 200 or the mouth of the user wearing the mouthpiece 200). The cavity 220 may be configured and/or located such that the inward movement of the film 203 does not interfere with positioning or location of the tongue.

In some embodiments, the film 203 may be connected to other locations of the first piece 201 and the second piece 202. For example, the film 203 may be moved backward or inward further toward the second ends of the first piece 201 and the second piece 202.

In some embodiments, the mouthpiece 200 may include a handle 210 configured to help improve extraction of the mouthpiece form the user's mouth, as illustrated in FIG. 1B. For example, the user may hold and pull the handle 210 to remove the mouthpiece 200 from the user's mouth. In some embodiments, the handle 210 may be placed on the film 203. In these and other embodiments, the handle 210 may be located such that the handle 210 does not interfere with movement of the film 103. For example, the handle 210 may not interfere with the folding and/or expanding of the film 203 as the mouthpiece 200 opens and closes. In these and other embodiments, the thickness of the handle 210 may be determined such that the handle 210 does not prevent the mouthpiece 200 from closing.

In some embodiments, the handle 210 may be located at other locations of the mouthpiece 200. For example, the handle 210 may be located on the first piece 201 or the second piece 202. In these and other embodiments, the handle 210 may be shaped and sized such that the handle 210 does not affect operations of the mouthpiece 200 or provide discomfort for the user.

In some embodiments, the handle 210 may be a part of the mouthpiece 200. For example, the handle 210 may be manufactured as a single piece including the first piece 201, the second piece 202, the film 203, and the handle 210. For example, the mouthpiece 200 may be manufactured using an injection-molding method in which the mold includes the first piece 201, the second piece 202, the film 203, and the handle 210.

In some embodiments, the handle 210 may be removably attached to the mouthpiece 100. For example, the handle 210 may be attached to the film 203 using adhesives such as resin, silicone, epoxy, etc. In these and other embodiments, the adhesives may be food-grade or non-toxic such that the adhesives are safe to use on in the mouthpiece 200. The handle 210 may be removed from the mouthpiece 200 by removing the adhesives. In other embodiments, any other suitable means of removably attaching the handle 210 to the mouthpiece 200 may be used.

Modifications, additions, or omissions may be made to the mouthpiece 200 without departing from the scope of the present disclosure. For example, in some embodiments, the mouthpiece 200 may include any number of other components that may not be explicitly illustrated or described.

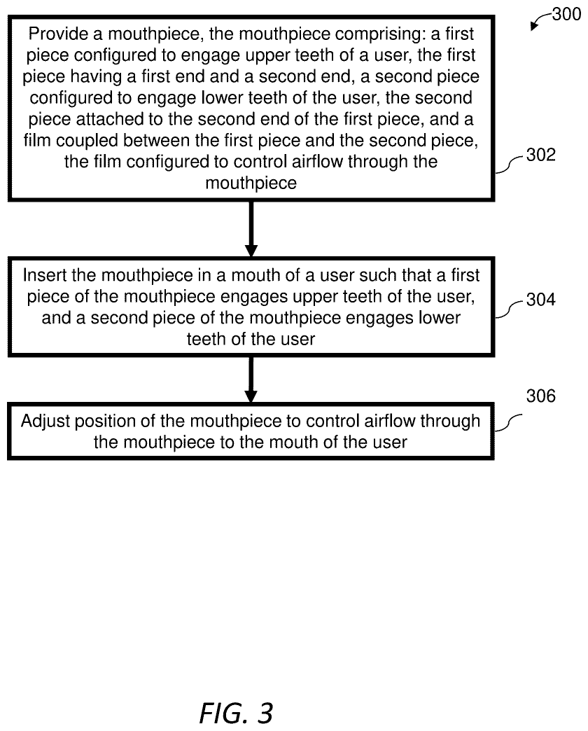

FIG. 3 illustrates a flow diagram of an example method 300 of reducing snoring, in accordance with one or more embodiments of the present disclosure. Although illustrated with discrete blocks, the steps and operations associated with one or more blocks of the method 300 may be divided into additional blocks, combined into fewer blocks, or eliminated, depending on the particular implementation.

In some embodiments, the method 300 may begin at block 302. At block 302, a mouthpiece may be provided. In some embodiments, the mouthpiece may correspond to the mouthpiece 100 and mouthpiece 200 of FIGS. 1A-1I and 2A-2C. For instance, the mouthpiece may include a first piece, a second piece, and a film, as described with respect to the mouthpiece 100 and the mouthpiece 200.

At block 304, the mouthpiece may be inserted into a mouth of a user such that the first piece of the mouthpiece engages upper teeth of the user, and the second piece of the mouthpiece engage lower teeth of the user. In some embodiments, the first piece and the second piece may be customized to fit the teeth of the user. Additionally or alternatively, the first piece and the second piece may include respective trays to engage the teeth of the user.

At block 306, the position of the mouthpiece may be adjusted to control the airflow through the mouthpiece. For example, the position of the mouthpiece may be verified and/or adjusted such that the mouthpiece, and particularly the film of the mouthpiece, covers the airway of the mouth.

Modifications, additions, or omissions may be made to the method 300 without departing from the scope of the present disclosure. For example, the outlined steps and operations are only provided as examples, and some of the steps and operations may be optional, combined into fewer steps and operations, or expanded into additional steps and operations without detracting from the essence of the disclosed embodiments.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. Additionally, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B” even if the term “and/or” is used elsewhere.

All examples and conditional language recited in the present disclosure are intended for pedagogical objects to aid the reader in understanding the present disclosure and the concepts contributed by the inventor to furthering the art, and are to be construed as being without limitation to such specifically recited examples and conditions. Although embodiments of the present disclosure have been described in detail, various changes, substitutions, and alterations could be made hereto without departing from the spirit and scope of the present disclosure.

What is claimed is:

1. A mouthpiece comprising:
a first piece configured to engage upper teeth of a user, the first piece having a first end and a second end;
a second piece configured to engage lower teeth of the user, the second piece attached to the second end of the first piece; and
a film coupled between the first piece and the second piece, the film configured to control airflow through the mouthpiece.

2. The mouthpiece of claim 1, wherein the film is configured to cover a gap between the first piece and the second piece.

3. The mouthpiece of claim 1, wherein the first piece, the second piece, and the film are manufactured from same type of material.

4. The mouthpiece of claim 3, wherein the material is silicone-like material.

5. The mouthpiece of claim 3, wherein the first piece, the second piece, and the film are molded as a single piece.

6. The mouthpiece of claim 1, wherein the film is further configured to operate as a spring mechanism to allow relative movement of the first piece and the second piece between a first position and a second position.

7. The mouthpiece of claim 6, wherein a thickness and curvature of the film is adjusted to require different levels of pressure to move between the first position and the second position, wherein the different levels of pressure help tighten soft tissues in a mouth of the user to open airway.

8. The mouthpiece of claim 1, wherein the film does not include any holes to block the airflow through the mouthpiece.

9. The mouthpiece of claim 1, wherein one or more of the film, the first piece, or the second piece includes one or more holes configured to control the airflow through the mouthpiece.

10. The mouthpiece of claim 1, wherein the film is further configured to stretch to adjust to size of mouth of the user.

11. The mouthpiece of claim 1, wherein the film is further configured to help keep the first piece and the second piece in position with relative to the upper teeth and the lower teeth of the user.

12. The mouthpiece of claim 1, wherein the film includes a curvature configured to provide space for tongue of the user.

13. The mouthpiece of claim 12, wherein the curvature is further configured to permit the film to be folded between the first piece and the second piece.

14. The mouthpiece of claim 1, further comprising a handle attached to the film, the handle configured to permit the user to move the mouthpiece relative to a mouth of the user.

15. The mouthpiece of claim 1, wherein the first piece and the second piece include extruded surfaces on surfaces coupled to the film.

16. The mouthpiece of claim 1, wherein the first piece and the second piece include one or more ridges configured to interlock the first piece and the second piece.

17. The mouthpiece of claim 1, wherein the film is configured to block the airflow through the mouthpiece to facilitate breathing through a nose of the user.

18. The mouthpiece of claim 1, wherein the mouthpiece complements continuous positive airway pressure (CPAP) or bilevel positive airway pressure (BiPAP) usage.

19. The mouthpiece of claim 1, wherein controlled airflow helps reduce snoring.

20. A method of reducing snoring comprising:
providing a mouthpiece, the mouthpiece comprising:
a first piece configured to engage upper teeth of a user, the first piece having a first end and a second end;
a second piece configured to engage lower teeth of the user, the second piece attached to the second end of the first piece; and
a film coupled between the first piece and the second piece, the film configured to control airflow through the mouthpiece;
inserting the mouthpiece in a mouth of a user such that a first piece of the mouthpiece engages upper teeth of the user, and a second piece of the mouthpiece engages lower teeth of the user; and
adjusting position of the mouthpiece to control airflow through the mouthpiece to the mouth of the user.
